(19) United States
(12) Patent Application Publication
(10) Pub. No.: US 2021/0186128 A1
(43) Pub. Date: Jun. 24, 2021

(54) MEDICAL PROCEDURE SHIELDING DEVICES AND GARMENTS

(71) Applicant: Mayo Foundation for Medical Education and Research, Rochester, MN (US)

(72) Inventor: Matthew T. Gettman, Byron, MN (US)

(73) Assignee: Mayo Foundation for Medical Education and Research, Rochester, MN (US)

(21) Appl. No.: 16/757,849

(22) PCT Filed: Nov. 14, 2018
(86) PCT No.: PCT/US2018/060978

§ 371 Date: Apr. 21, 2020

Related U.S. Application Data
(60) Provisional application No. 62/585,643, filed on Nov. 14, 2017.
(60) Provisional application No. 62/617,961, filed on Jan. 16, 2018.

(51) Int. Cl. A41D 13/12; A61B 46/00; A61B 46/23; A61B 46/27

(52) U.S. Cl. CPC A41D 13/1254; A61B 46/30; A61B 2046/205; A61B 46/27; A61B 46/23

(57) ABSTRACT

Shielding devices can be used during medical procedures. For example, this document describes drape-like devices that can be attached to an endoscope shaft, to a primary drape, or directly to the patient. The drape-like devices promote patient privacy during procedures that access the patient's body orifices. Garments are also described that promote patient privacy and patient protection during procedures, examinations, medical events like childbirth that involve access the patient's body orifices such as urogenital and anal orifices.

### CROSS-REFERENCE TO RELATED APPLICATIONS

This application claims priority to U.S. Application Serial No. 62/617,961, filed on Jan. 16, 2018 and 62/585,643, filed on Nov. 14, 2017. The disclosures of the prior applications are considered part of the disclosure of this application, and are incorporated in their entirety into this application.

### BACKGROUND

### 1. Technical Field

This document relates to shielding devices and garments used during medical procedures, medical examinations on the urogenital and anal organs, and during childbirth. This document relates to devices and garments that can promote patient privacy, enhance modesty, and provide patient protection yet not impede medical care. For example, this document relates to drape-like devices that can be attached to an endoscope shaft or to a primary surgical drape, and that promote patient privacy and patient protection during procedures, examinations and medical events that require access the patient's body orifices such as urogenital and anal orifices.

### 2. Background Information

Having procedures and interventions, examinations, and events performed on or occurring with the urogenital and colorectal organs can cause considerable stress for patients. Unintended trauma can also potentially occur to these unshielded organs during the above situations. Having urogenital and colorectal organs exposed during interventions, examinations, and medical events with these organs can also be a potential for medical legal risk for medical institutions. In many instances these interventions arcdone with endoscopes or probes. One stress for patients is the fact that their genitalia and anal area may be exposed when undergoing interventions, examinations, or medical events involving these organs.

### SUMMARY

This document describes shielding devices and garments used during medical procedures, during medical examinations and during medical events such as childbirth. For example, this document describes drape-like devices that can be attached to an endoscope shaft or to a primary surgical drape, or worn by the patient with goals of promoting patient privacy and patient protection during procedures, examinations, or medical events like childbirth that require access to the patient's body orifices such as urogenital and anal orifices.

In one aspect, this disclosure is directed to a shielding device adapted for use during a medical procedure. Such a shielding device can include: (i) a shield body defining a proximal opening and a distal opening; and (ii) an attachment area coupled to the shield body near the distal opening, the attachment area configured for releasably attaching the shielding device to a drape or gown.

Such a shielding device may optionally include one or more of the following features. The shield body may be conical or frustoconical. The shield body may be cylindrical. The shielding device may also include a mechanism for selectively closing the proximal opening. The attachment area may include an adhesive.

In another aspect, this disclosure is directed to another shielding device adapted for use during a medical procedure. Such a shielding device includes: (a) a shield body defining a proximal opening and a distal opening; (b) an attachment mechanism disposed at the proximal opening, the attachment mechanism operable to releasably couple the shielding device to a shaft of a medical instrument; and (c) an attachment area coupled to the shield body near the distal opening.

Such a shielding device may optionally include one or more of the following features. The shield body may be conical or frustoconical. The shield body may include contoured lateral cut-outs. The shielding device may include an umbrella mechanism attached to the shield body to make the shielding device radially expandable and retractable. The shield body may be circular. The shield body may include a plurality of generally triangular portions that slide with respect to each other like a fan.

In another aspect, this disclosure is directed to a modular medical privacy garment that includes: (1) a modular base garment with a waistband and two leg holes, the base garment defining an opening; and (2) a modular component that is attachable to the base garment to cover the opening, the modular component defining one or more openings.

Such a modular medical privacy garment may optionally include one or more of the following features. The modular medical privacy garment may also include: (3) an aseptic preparation kit that is integrated with the base garment; and (4) a cleanup kit that is integrated with the base garment. The modular component may be shaped like a box. A peripheral wall of the box may be pleated to allow extension and retraction of the box.

Particular embodiments of the subject matter described in this document can be implemented to realize one or more of the following advantages. In some embodiments, the devices and garments described herein can improve patient experience by providing better patient privacy/modesty. Additionally, in some cases the devices and garments provided herein can advantageously provide protection of the areas being instrumented. This advantage has direct benefit for the patient but also is a means that medical institutions can use to mitigate medicolegal claims and reduce risk. In some embodiments, thermoregulation of the patient can be facilitated using the devices and garments described herein. In some embodiments, protection from ionizing radiation can be achieved. Moreover, in some embodiments enhanced image guidance can be advantageously provided using the devices described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of an example conical shielding device in accordance with some embodiments provided herein.

FIG. 2 is a perspective view of an example cylindrical shielding device in accordance with some embodiments provided herein.

FIG. 3 is a perspective view of an example semi-spherical shielding device in accordance with some embodiments provided herein.

FIG. 4 is a perspective view of an example trough-shaped shielding device in accordance with some embodiments provided herein.

FIG. 5 is a cross-sectional view of an example shielding device showing a first example style of an entry zone closure.

FIG. 6 is a cross-sectional view of an example shielding device showing a second example style of an entry zone closure.

FIG. 7 is a cross-sectional view of an example shielding device showing a third example style of an entry zone closure.

FIG. 8 is a perspective view of a cystoscope with an attached example conical shielding device in accordance with some embodiments provided herein.

FIG. 9 is a plan view of an example rectangular shielding device in accordance with some embodiments provided herein.

FIG. 10 is a plan view of an example expandable circular shielding device in accordance with some embodiments provided herein.

FIG. 11 is a longitudinal cross-sectional view of an example conical shielding device with lateral cutouts to accommodate a patient's anatomy.

FIG. 12 is a plan view of the shielding device of FIG. 11.

FIG. 13 is a longitudinal cross-sectional view of an example semi-spherical shielding device with lateral cutouts to accommodate a patient's anatomy.

FIG. 14 is a plan view of the shielding device of FIG. 13.

FIG. 15 is a plan view of a shielding device showing an example mechanism for attachment of the shielding device to a primary patient drape.

FIG. 16 is a plan view of a shielding device showing another example mechanism for attachment of the shielding device to a primary patient drape.

FIG. 17 is a plan view of a shielding device showing another example mechanism for attachment of the shielding device to a primary patient drape.

FIG. 18 is a plan view of a shielding device showing another example mechanism for attachment of the shielding device to a primary patient drape.

Like reference numbers represent corresponding parts throughout.

FIG. 19 is a plan view of a male urologic shielding garment that is preferentially used during procedures and examinations on the penis and scrotum

FIG. 20 is a side view of the garment of FIG. 19 in a closed configuration.

FIG. 21 is a side view of the garment of FIG. 19 in an open configuration.

FIG. 22 is a plan view of a shielding garment that is preferentially used during procedures and examinations of the anal area. The garment is shown in a closed configuration.

FIG. 23 is a plan view of the garment of FIG. 22 shown in an open configuration.

FIG. 24 is a side view of the garment of FIG. 22 shown in a closed configuration.

FIG. 25 is a side view of the garment of FIG. 22 shown in an open to configuration.

FIG. 26 is a plan view of a shielding garment that also incorporates an aseptic preparation kit and a cleanup kit with the garment.

FIG. 27 shows a component of a modular shielding garment called the cystoscopy base garment.

FIG. 28 shows a component of a modular shielding garment called the colonoscopy base garment.

FIG. 29 shows another component of the modular shielding garments called a wraparound flap component.

FIG. 30 shows another component of the modular shielding garments called a skirt flap component.

FIG. 31 shows another component of the modular shielding garments called a split skirt flap component.

FIG. 32 shows another component of the modular shielding garments called a raised button design component.

FIG. 33 shows a plan view of another component of the modular shielding garments called an expandable accordion design component.

FIG. 34 shows a side view of the expandable accordion design component of FIG. 33.

FIG. 35 shows another component of the modular shielding garments called an adjustable diaphragm design component. The opening size is adjusted to a small opening configuration.

FIG. 36 shows the adjustable diaphragm design component of FIG. 35 with the opening size adjusted to a large opening configuration.

FIG. 37 shows a plan view of an example modular shielding garment and associated components used for specific access to the female urethra and vaginal area

FIG. 38 shows an example modular shielding garment and associated components used specifically for childbirth.

### DETAILED DESCRIPTION

This document describes shielding devices and garments that are used during medical procedures, medical examinations and medical events such as childbirth. For example, this document describes drape-like devices that can be attached to an endoscope shaft, to a primary drape, or directly to the patient. The document also describes garments that can be worn by the patient to provide shielding with privacy and protection during medical procedures, medical examinations and during medical events like childbirth. Collectively these inventions promote patient privacy and patient protection during procedures, examinations, medical events like childbirth that involve access the patient's body orifices such as urogenital and anal orifices.

In some embodiments, the devices and garments described herein permit enhanced privacy for a patient undergoing any intervention, and also provide personal protection and enhanced comfort. A theme of these devices and garments is that the surgeon or clinician would have unimpeded access to the orifice of interest, but at the same time there would be offered to the patient the maximum means to conceal the organs of interest as well during the intervention. When the medical procedure, medical examination or medical event is not occurring the devices and garments describe would essentially completely conceal the urogenital and anal areas.

In some embodiments, basic components or the devices and garments include the following segments: shielding cone (or other shape), cone supports, cone fixators/base ring anchoring mechanism, and cone entry zone. The size and configuration of the cone (or other shape) would be scalable and related to the orifice undergoing intervention. Different types of shapes would include, but are not limited to, such as a pyramid, cylinder, half-pipe/trough, bowl/cup, and peanut-shape design. Cone supports include, but are not limited to, fabric, polymer and ribbed design, malleable mesh design, metallic supports with struts and ring design. Cone fixators to the base ring design would include, but are not limited to, both direct to patient and indirect to patient designs. Fixators in either design can include fabric, mesh, elastic bands, adhesive tabs, fixation to purpose build garment, fixation to waistband, hook and loop, ties with suture or string, wire twist ties, snaps, hooks, tethers to indwelling catheters, or attachment to surgical drapes and retractors. The cone entry zone would also be tied to the type of intervention being performed. There would be many different types of designs to impact the opening and closure of the cone with snaps, hook and loop. malleable opening, clips, magnets, and spring-loaded mechanisms to provide some examples.

While one intended use is for when intervening on the urogenital and anal openings, the concepts would pertain to a means to increase privacy of intervention and patient comfort for any surgical field or intervention on any bodily orifice or anatomic region.

Having procedures and interventions, examinations, and medical events like childbirth involving the urogenital and colorectal organs can cause considerable mental stress for patients. This is especially the case when the procedures are done in awake fashion. There is also considerable mental stress for patients that are also undergoing procedures under general anesthesia because they know their genitalia will be exposed during these events. This is also a concern that during these interventions in any setting that unshielded urogenital and anorectal organs can be harmed during the procedures. As such interventions on these organs has associated potential for medicolegal risk related to harm of the urogenital and anal organs or related to breaches in patient exposure. In many instances, these interventions arc done with endoscopes or probes yet the same concerns exist when patients are undergoing other surgical techniques, examinations on these organs, or during childbirth. One stress for patients is the fact that their genitalia and anal area may be exposed when undergoing such interventions. The devices and garments described herein are used to shield the genitals and anal area during interventions, yet not impede the visualization of the field during the interventions. The devices and garments would permit patients to feel less vulnerable during these sensitive interventions and feel more confident that these organs will not be injured during the procedure, thereby improving the patient experience.

The devices and garments described herein may also permit belier protection of the areas being instrumented, and may have a role in thermoregulation of the patient, and may prevent exposure to ionizing radiation. Moreover, proposed components in some embodiments of the invention may have a role in image guidance and endoscope tracking. The devices and garments that are proposed for shielding would have one or more of the major components including the endoscope or probe fixation zone, the screen or privacy barrier component, the drape fixation zone, the patient fixation zones, heating and cooling elements, and visualization screens/navigation systems.

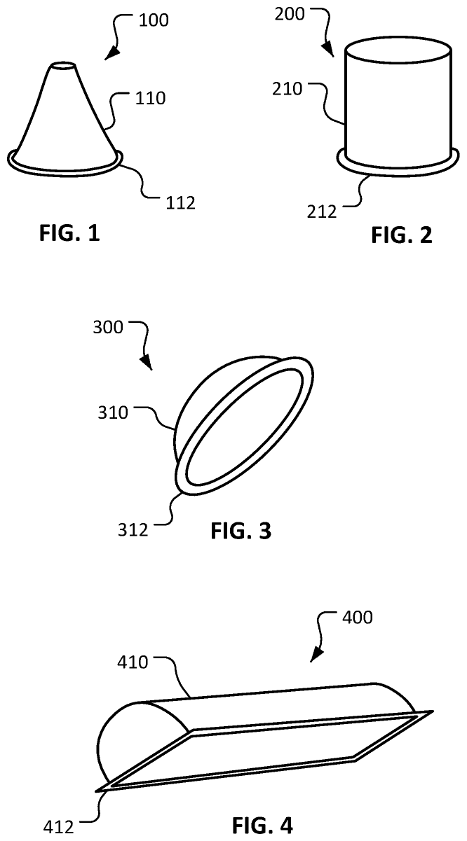

Referring to FIG. 1, an example conical shield 100 can be used to provide privacy for a patient undergoing, or associated with, procedures and/or interventions performed on the patient's urogenital and colorectal organs while allowing a surgeon or clinician to have unimpeded access to the orifice or organ of interest. Conical shield 100 includes a frustoconical body 110 and an attachment area 112 at a distal end of frustoconical body 110.

In some cases, such as in the depicted embodiment, the small end (the proximal end) of frustoconical body 110 is open so as to provide unimpeded access to the patient's orifice or organ of interest. Hence, the small end of frustoconical body 110 can be referred to as the cone-entry-zone. In some cases, the small end of frustoconical body 110 is selectively openable and closeable as described further below. The distal end (larger end) of the frustoconical body 110 is also open to provide patient access.

The attachment area 112 can be used to releasably secure conical shield 100 to a base such as a primary drape, a waistband, clothing, or directly to the patient's skin. For example, in some cases attachment area 112 can be stitched to a primary drape, or other attachment techniques can be used such as adhesives (e.g., using a removable liner), hook and loop fasteners, snaps, hooks, clamps, tethers, and the like. In some cases, conical shield 100 can be incorporated into a primary surgical drape.

Conical shield 100 can be a sterile item or a non-sterile item. In some embodiments, conical shield 100 is a single-use, disposable item. In some embodiments, conical shield 100 is a reusable item that can be laundered. Conical shield 100 can be made of materials such as, but not limited to, polypropylene, polyethylene, cotton, and the like.

In some cases, frustoconical body 110 is made of reinforced material. For example, plastic ribs can be attached to, laminated within, or woven within the material of frustoconical body 110. In some cases, metallic ribs can be used. Such metallic ribs can be malleable in some cases. A mesh material can be included in frustoconical body 110 in some cases. Such reinforcing materials can be used to help frustoconical body 110 maintain its conical shape. In some cases, frustoconical body 100 is made of an inherently stiff material, and thereby maintains its shape.

Referring to FIG. 2, an example cylindrical shield 200 can be used to provide privacy for a patient undergoing, or associated with, procedures and/or interventions performed on the patient's urogenital and colorectal organs while allowing a surgeon or clinician to have unimpeded access to the orifice or organ of interest. Cylindrical shield 200 includes a cylindrical body 210 and an attachment area 212. Each of the proximal end (the top as shown in FIG. 2) and the distal end (the bottom as shown in FIG. 2) of cylindrical shield 200 are open.

Cylindrical shield 200 can include any of the features and constructions described above in reference to conical shield 100.

Referring to FIG. 3, an example semi-spherical shield 300 can be used to provide privacy for a patient undergoing, or associated with, procedures and/or interventions performed on the patient's urogenital and colorectal organs while allowing a surgeon or clinician to have unimpeded access to the orifice or organ of interest. Semi-spherical shield 300 includes a semi-spherical body 310 and an attachment area 312.

Semi-spherical shield 300 can include any of the features and constructions described above in reference to conical shield 100.

Referring to FIG. 3, an example trough-shaped shield 400 can be used to provide privacy for a patient undergoing, or associated with, procedures and/or interventions performed on the patient's urogenital and colorectal organs while allowing a surgeon or clinician to have unimpeded access to the orifice or organ of interest. Trough-shaped shield 400 includes a trough-shaped body 410 and an attachment area 412.

Trough-shaped shield 400 can include any of the features and constructions described above in reference to conical shield 100.

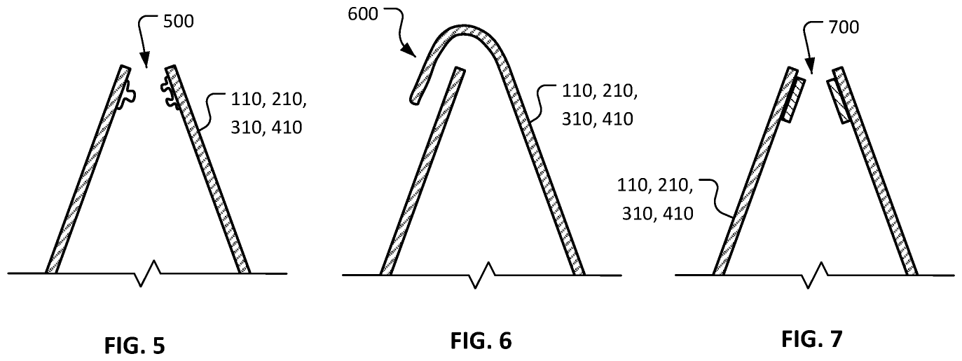

Referring to FIGS. 5-7, various types of mechanisms are shown that can be used to make the open ends of the shield devices described herein to be selectively openable and closeable. For example, in FIG. 5, a snap mechanism 500 is shown. One or more such snap mechanisms 500 can be included in a single shield device.

In FIG. 6, an overlapping configuration 600 is depicted. Accordingly, the shield device includes enough material such that the open end can be covered/closed and readily opened by simply moving the overlapping material. In some cases, a closure can be included such as adhesive, hook and loop, magnets, snaps, a button, and the like.

In FIG. 7, a hook and loop closure 700 is depicted. This may also be a magnetic closure.

Other types of closure mechanisms are also envisioned within the scope of this disclosure. Such other types of closure mechanisms can include, but are not limited to, clips, pinch spring mechanisms, malleable members, and the like.

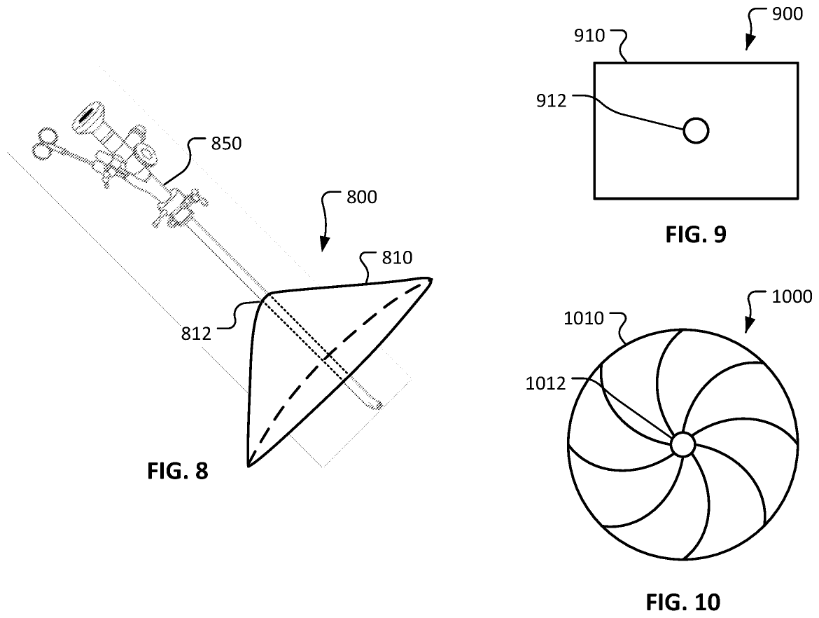

Referring to FIG. 8, some embodiments of the shielding devices described herein can be configured to releasably attach to a medical instrument such as a cystoscope, colonoscope, endoscope, and other probes. For example, a conical shield 800 can be releasably coupled to a shaft of a cystoscope 850.

Conical shield 800 includes a frustoconical body 810 and a proximal opening 812 through which the shaft of cystoscope 850 extends. Conical shield 800 can include any of the features and constructions described above in reference to conical shield 100.

Opening 812 can include various types of mechanisms for releasably coupling with the shaft of cystoscope 850. For example, mechanisms can be included such as, but not limited to, adhesive/tape, a rubber sleeve/grommet, a clip, a snap, a tie, a twist tie, a cable tie, and the like.

In some embodiments, conical shield 800 is selectively radially collapsible and expandable like an umbrella. An umbrella mechanism can be coupled to conical shield 800.

Referring to FIG. 9, a rectangular shield 900 can be configured to releasably attach to a medical instrument in the manner described above in reference to conical shield 800. Rectangular shield 900 includes a rectangular body 910 and an opening 912 through which a shaft of a medical instrument (e.g., cystoscope 850) can extend. Rectangular shield 900 can include any of the features and constructions described above in reference to conical shields 100 and 800.

Referring to FIG. 10, a circular shield 1000 can be configured to releasably attach to a medical instrument in the manner described above in reference to conical shield 800. Circular shield 1000 includes a circular body 1010 and an opening 1012 through which a shaft of a medical instrument (e.g., cystoscope 850) can extend. Circular shield 1000 can include any of the features and constructions described above in reference to conical shields 100 and 800. In the depicted embodiment, circular shield 1000 is expandable and collapsible like a fan. That is, circular shield 1000 includes a plurality of generally triangular portions that are slidable relative to each other between forming an expanded circular shape (as shown) or a collapsed shape (e.g., like a pie-shaped segment of a circle).

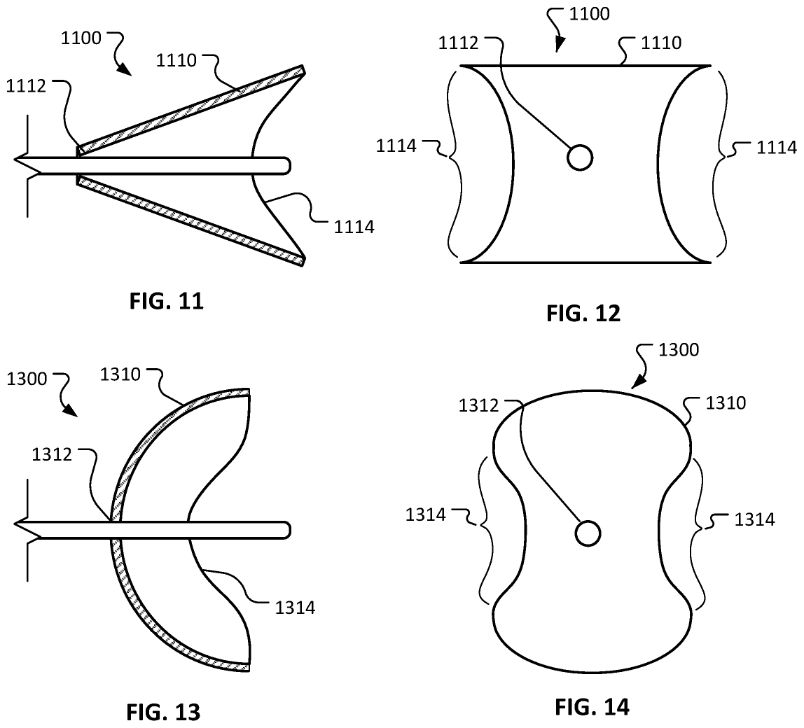

Referring to FIGS. 11 and 12, some shielding devices described herein can be shaped to conform with a patient's anatomy. FIG. 11 is a longitudinal cross-sectional view of an example conical shielding device 1100 with lateral cutouts 1114 to accommodate a patient's anatomy. FIG. 12 is a plan view of conical shielding device 1100.

Conical shield 1100 can be configured to releasably attach to a medical instrument in the manner described above in reference to conical shield 800. Conical shield 1100 includes a conical body 1110 and an opening 1112 through which a shaft of a medical instrument (e.g., cystoscope 850) can extend. Conical shield 1100 can include any of the features and constructions described above in reference to conical shields 100 and 800. Conical shield 1100 also defines lateral cutouts 1114 which are contoured to interface with a patient's anatomy such as legs of the patient.

Referring to FIGS. 13 and 14, additional shielding devices described herein can be shaped to conform with a patient's anatomy. FIG. 13 is a longitudinal cross-sectional view of an example semi-spherical shielding device 1300 with lateral cutouts 1314 to accommodate a patient's anatomy. FIG. 14 is a plan view of semi-spherical shielding device 1300.

Semi-spherical shield 1300 can be configured to releasably attach to a medical instrument in the manner described above in reference to conical shield 800. Semi-spherical shield 1300 includes a semi-spherical body 1310 and an opening 1312 through which a shaft of a medical instrument (e.g., cystoscope 850) can extend. Semi-spherical shield 1300 can include any of the features and constructions described above in reference to conical shields 100 and 800. Semi-spherical shield 1300 also defines lateral cutouts 1314 which are contoured to interface with a patient's anatomy such as legs of the patient.

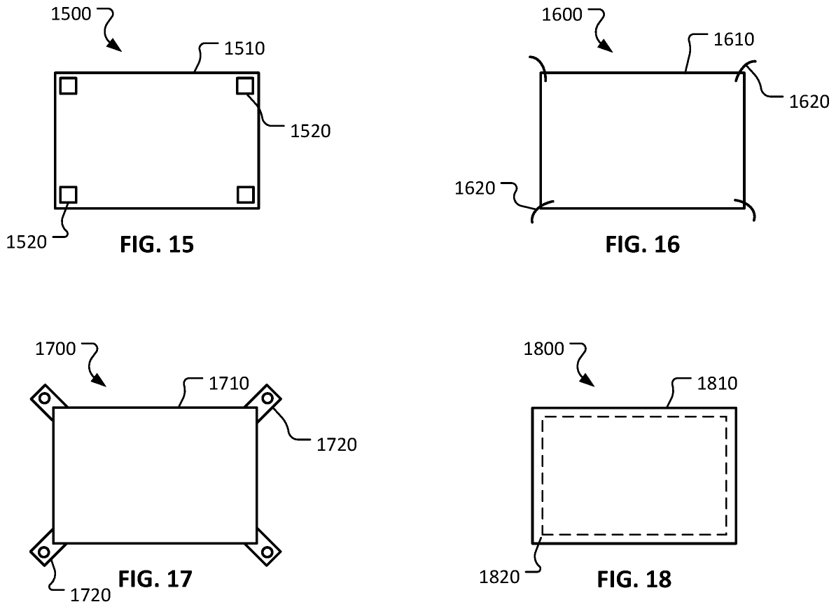

Referring to FIGS. 15-18, some of the shielding devices described herein can be configured to be releasably attached to other articles such as, but not limited to, drapes or gowns. While the shielding devices depicted in FIGS. 15-18 are rectangular, it should be understood that any of the shielding device shapes and configurations described herein can be configured to be releasably attached to other articles. To that end, in some cases such shielding devices can include an opening for engaging with the shaft of a medical instrument such as a probe.

Shielding device 1500 includes a body 1510 and attachment elements 1520 that can be weights, magnets, hook and loop elements, and the like. Shielding device 1600 includes a body 1610 and attachment elements 1620 that can be ties, pins, clasps, sutures, and the like. Shielding device 1700 includes a body 1710 and attachment elements 1720 that can be snaps, buttons, button holes, and the like. Shielding device 1800 includes a body 1810 and attachment element 1820 that can be one or more adhesive areas.

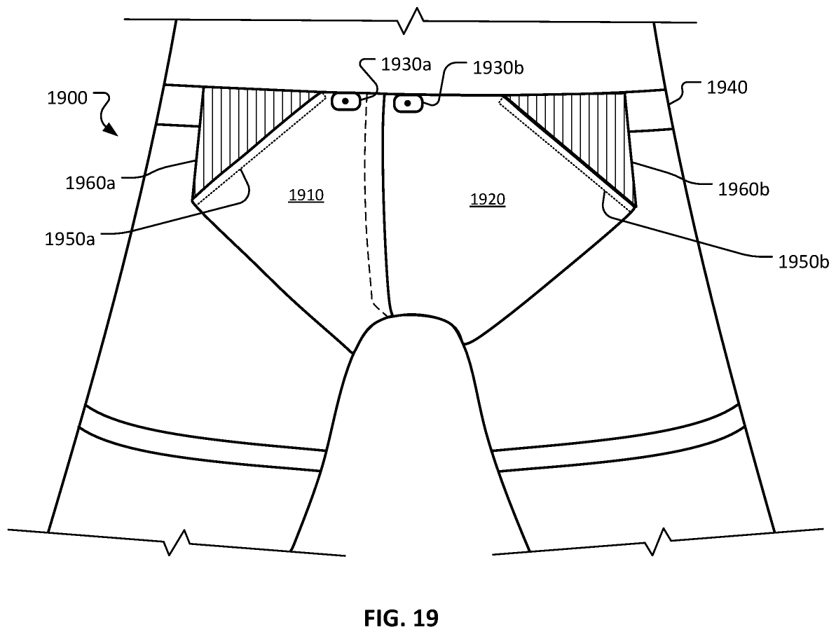

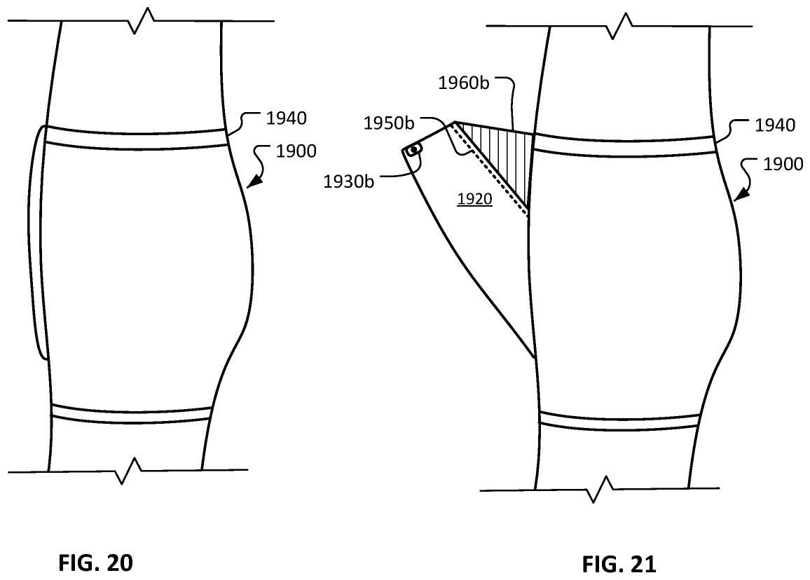

FIG. 19 illustrates an example male urologic shielding garment 1900 that is designed for interventions and examinations of the penis and scrotum. With this embodiment, included are a right side genital flap 1910 and a left side genital flap 1920 that are connected via clasping mechanisms 1930a and 1930b, respectively, to the elastic waistband 1940 of the garment 1900. Each genital flap is affixed to the fabric of the garment 1900 along the bottom and side edges. In some embodiments, elastic panels 1960a-b are included to facilitate expansion/contraction of the genital flaps 1910 and 1920 during opening and closing. In some embodiments, there are a spring mechanisms 1950a-b that keep the flaps 1910 and 1920 in an open position after deployment. Such spring mechanisms 1950a-b (e.g., leaf springs) can be biased to the opening configuration. With this embodiment, the genital flaps 1910 and 1920 are unclasped at the time the medical intervention or examination on the male genitalia is set to occur. At the completion of the event, the genital flaps 1910 and 1920 are reaffixed to the elastic band 1940 using the clasps 1930a-b on the genital flaps 1910 and 1920. FIG. 20 shows a sagittal view of the undeployed (closed) garment 1900, and FIG. 21 shows a sagittal view of the deployed (open) garment 1900.

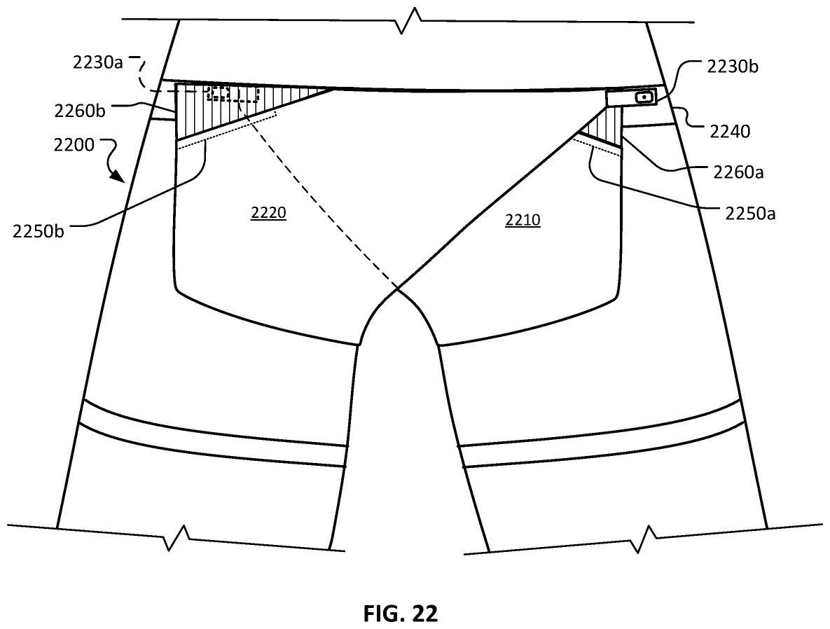

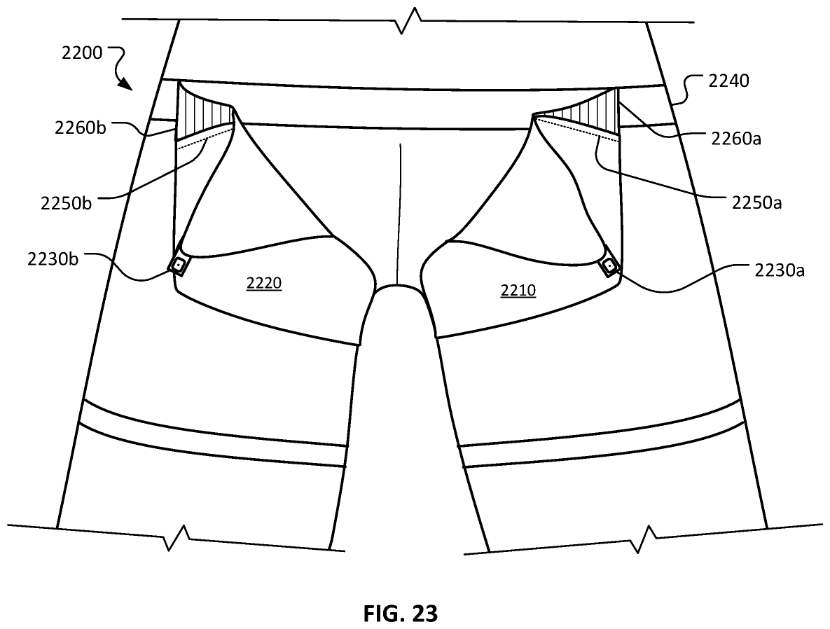

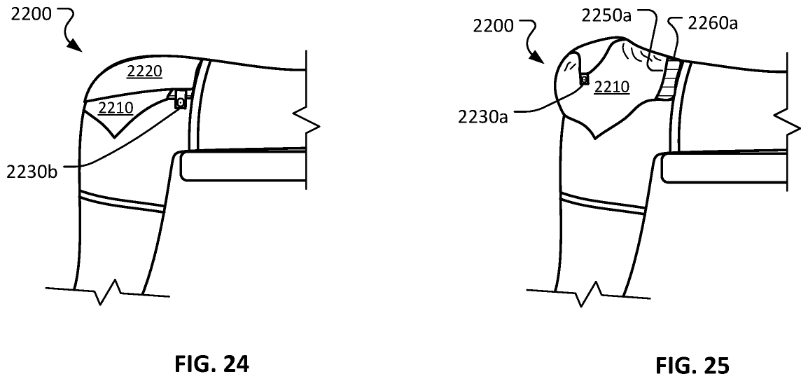

FIG. 22 illustrates an example anal shielding garment 2200 that is designed for interventions and examinations of the anus in both men and women. With this embodiment, included are right 2210 and left 2220 anal flaps that are similarly connected via a clasping mechanisms 2230a-b, respectively, to the elastic waistband 2240 of the garment 2200. Each anal flap 2210 and 2220 is affixed to the fabric of the garment 2200 and, in some embodiments, there are respective spring mechanisms 2250a-b that keep the flaps 2210 and 2220 in position after deployment (opening). With this embodiment, the anal flaps 2210 and 2220 are unclasped at the time the medical intervention or examination on the anal areas is set to occur. Elastic panel portions 2260a-b expand/contract in response to the opening and/or closing of the anal flaps 2210 and 2220. At the completion of the medical event, the anal flaps 2210 and 2220 are reaffixed to the elastic band 2240 using the clasps 2230a-b on the anal flaps 2210 and 2220. FIG. 23 shows a posterior view of the garment 2200 with the anal flaps 2210 and 2220 deployed open and the working space created to the anus. FIG. 24 shows a side view of the undeployed anal shielding garment 2210 and 2220 and FIG. 25 shows a side view of the deployed anal garment 2200 and access created to the anus by the deployed anal flaps 2210 and 2220.

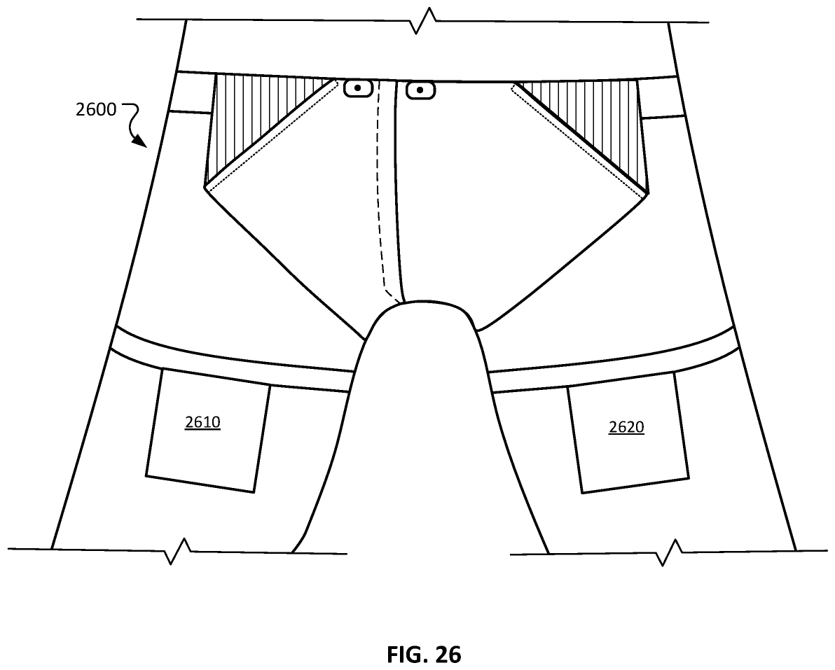

FIG. 26 illustrates an example shielding garment 2600 with an incorporated aseptic preparation kit 2610 and an incorporated cleanup kit 2620. Such kits 2610 and 2620 can be incorporated with any of the garments described herein. With use of the shielding garment 2600, the need for having separate drapes for the planned intervention is eliminated. In essence, the person is now wearing the drape when they enter the environment for their care. If a man were coming in for a cystoscopic examination, for example, they would be asked to remove their underwear and put on the shielding garment 2600 in place of the underwear. Attached to the shielding garment 2600 would be the aseptic preparation kit 2610. After the attendant would position the patient, the attendant in this example would deploy the genital flaps, complete the aseptic preparation of the genital area and then reaffix the flaps. At the time of the cystoscopy, the flaps would then be deployed and the cystoscopic examination would be performed. At the end of the procedure, the attendant would use the cleaning kit 2620 (affixed to the garment) to clean off the genitalia before getting the patient off the examination table. In some embodiments, kits 2610 and 2620 can be pockets sewn into the garment 2600. In some embodiments, kits 2610 and 2620 can be detachable from the garment 2600 (e.g., by tearing off a perforated attachment portion, by a pressure-sensitive adhesive, by hook-and-loop fastening, and the like).

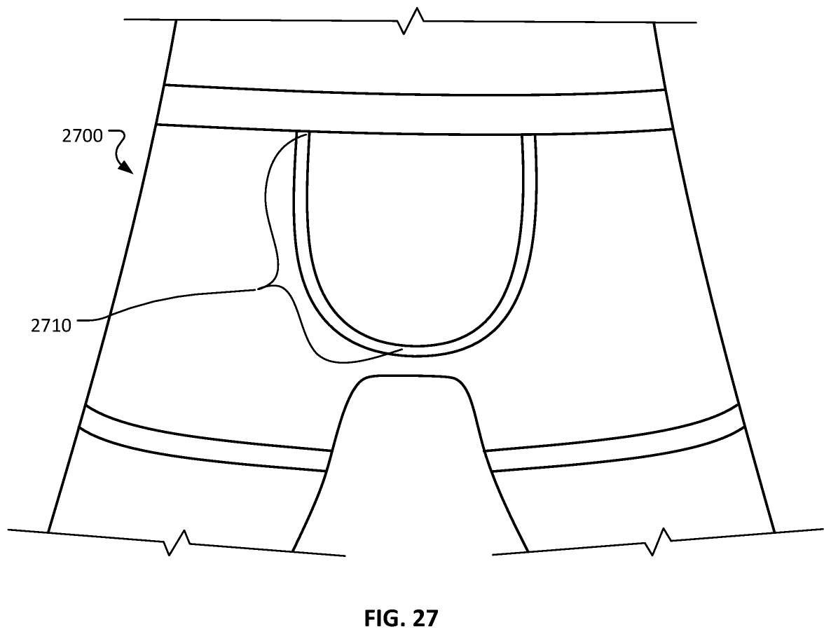

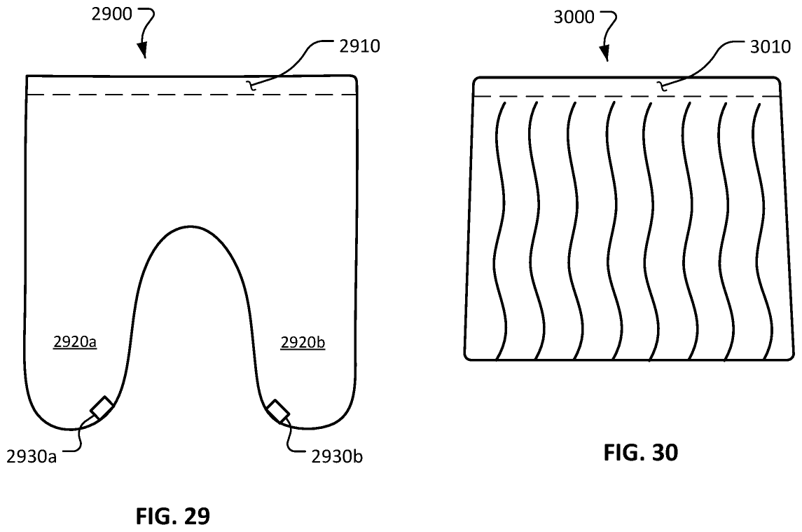

FIG. 27 illustrates an example modular shielding garment 2700 in one embodiment called a modular base cystoscopic garment. Modular shielding garment 2700 includes a waistband, and defines two leg holes. Modular shielding garment 2700 also defines an opening 2710. The illustration demonstrates such an opening 2710 created in the anterior superior region of the shielding garment 2700, as would be useful for cystoscopy (as an example). Using the modular base garment 2700, additional components of varying designs can then be used in conjunction with the modular base garment 2700 to fulfill the needs of the shielding garment for the patient's gender and the type of procedure to be performed, for example. That is, a modular component is attachable to the base garment 2700 to cover opening 2710. In some embodiments, the modular component can also define one or more openings. FIGS. 29-39 provide examples of such modular components.

Of note, base garment designs are also included in the scope of this invention for access to the female vaginal area. In the base female garment design, the similar principles are used and the window created in the garment is located in the anterior region as well but more inferior than the base cystoscopic garment.

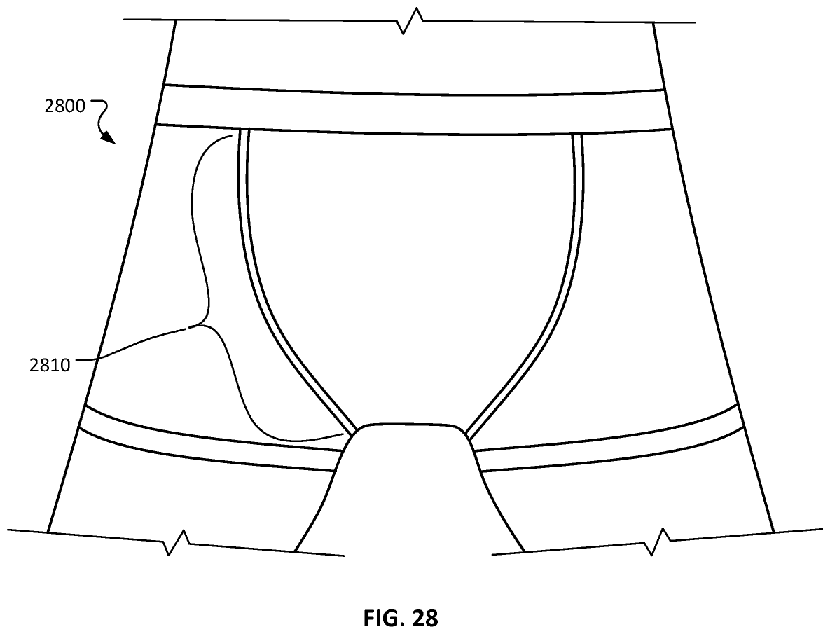

FIG. 28 illustrates another example modular shielding garment 2800, called a modular base colonoscopic garment. This demonstrates the window 2810 created in the posterior region of the shielding garment 2800 that would be required for anal interventions and examinations (as an example). Using the modular base colonoscopic garment 2800, additional components of varying designs are then used in conjunction with the modular base garment 2800 to fulfill the needs of the shielding garment.

FIG. 29 illustrates a modular shielding garment component 2900 that can be used in conjunction with the modular base garment design concept (e.g., modular base garment 2700 and/or 2800). In this particular embodiment, an edge portion of the fabric 2910 can be releasably attached to the elastic waistband of the modular base garment to create a hinged drape type of design. This drape of fabric includes two portions 2920a and 2920b that can be wrapped circumferentially around the endoscope to enhance coverage during an endoscopic intervention as an example. Each portion 2920a-b can include a respective closure mechanism 2930a-b that can be used to secure the portions 2920a-b around the endoscope. The closure mechanisms 2930a-b can be magnets, hook-and-loop, malleable elements, adhesive elements, and the like.

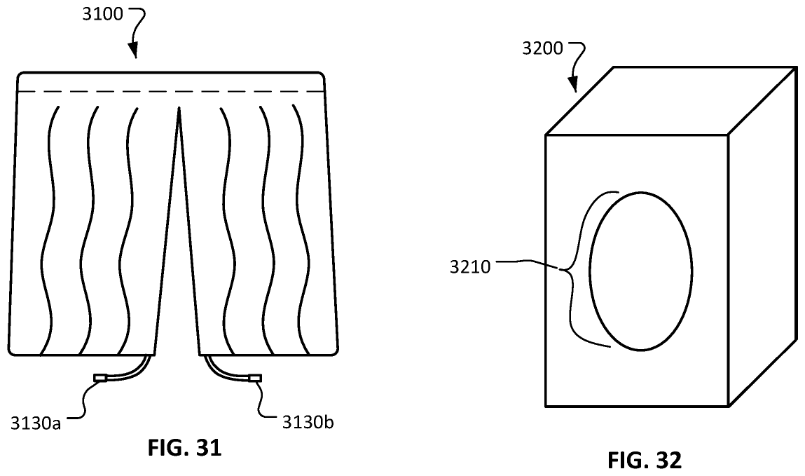

FIG. 30 illustrates a modular shielding garment component 3000 that can be used in conjunction with the modular base garment design concept (e.g., modular base garment 2700 and/or 2800). In this particular embodiment, an edge portion of the fabric 3010 can be releasably attached to the elastic waistband of the modular base garment. In this modular component, a fabric is similarly used to create a hinged drape design in a skirt type configuration with an option to have a divided fabric design 3100 as shown in FIG. 31. In some embodiments, one or more openings can be included within the fabric. In some embodiments, closure mechanism 3130a-b that can be used to secure the portions 3120a-b around the endoscope can be included. The closure mechanisms 3130a-b can be magnets, hook-and-loop, malleable elements, adhesive elements, and the like.

In other embodiments other modular components could be used with the modular base garment design (e.g., modular base garment 2700 and/or 2800) to achieve the goals of the inventions. These components described could be used in conjunction with any of the base garment designs. Examples include a raised button to design 3200 as shown in FIG. 32. In this example that is shaped like a box, the raised button design 3200 includes an ovular opening 3210. Openings of any size and shape can be included.

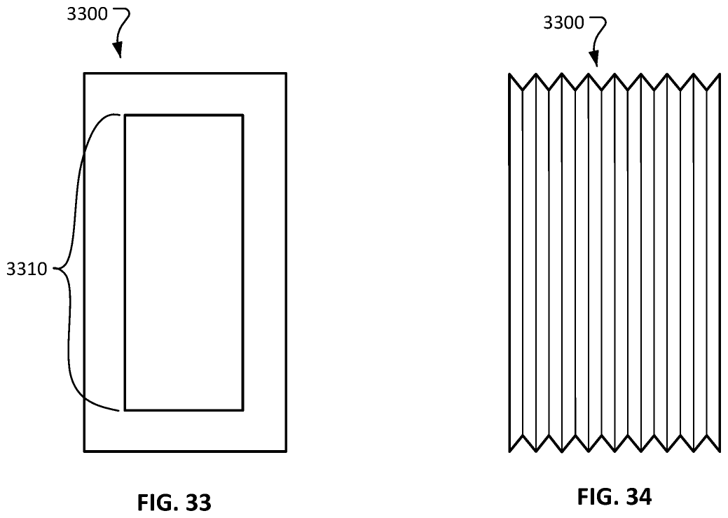

As seen in the anterior-posterior view of FIG. 33 and in the side view of FIG. 34, the modular components can include an expandable accordion design 3300 that defines an opening 3310. In this example, the peripheral wall of the design 3300 includes pleats.

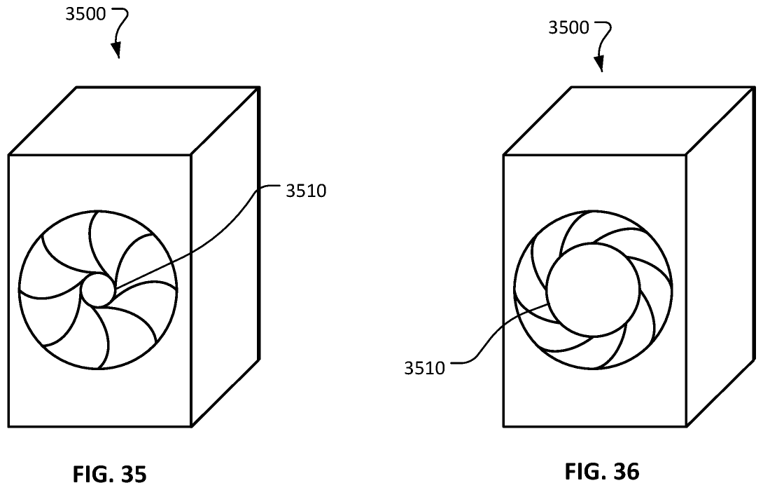

In another example, as shown in FIGS. 35 and 36, the modular components can include design 3300 that defines an opening 3500 that has an opening 3510 that is adjustable in size (e.g., diameter).

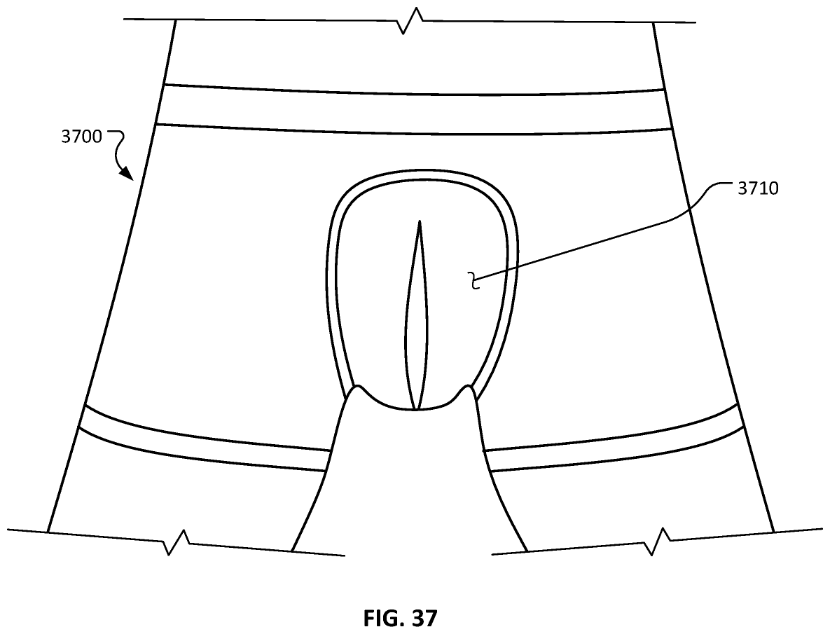

FIG. 37 illustrates a garment 3700 that would be used for procedures and examinations of the vaginal area, specifically pelvic examinations. This shielding garment 3700 would have as one component a modular base garment with a window included anteriorly and inferiorly to facilitate access to the vaginal area. Incorporated to the base garment in this embodiment would be affixed a button design 3710 with a vertical opening to facilitate privacy for the pelvic examination. With this embodiment, the patient would exchange her underwear for this garment 3700 before the pelvic examination and preparation and cleaning equipment could be included with the garment 3700 (as depicted in FIG. 26).

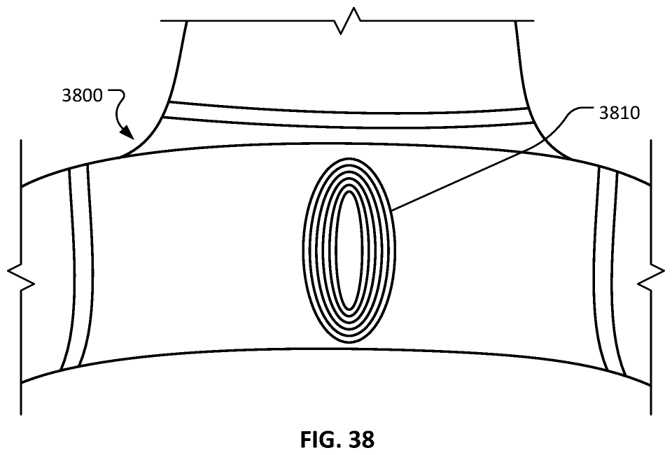

FIG. 38 illustrates a garment 3800 that would be used during childbirth. With this embodiment, the patient would be asked to remove their underwear and put on this garment 3800 upon arrival for obstetric care. This garment 3800 would again use a base garment design with a larger window proving access to the vaginal area. In the preferred embodiment, the garment would include a raised accordion design 3810 that provides the shielding of the genitalia. This design would permit the patient to be shielded at all time during the delivery process with the vagina only visible to the care provider helping with the labor and delivery. For this embodiment in particular, there would be a means to use elements of thermoregulation of the garment during the labor and delivery process. During the laboring process, for example, heating of the garment may facilitate comfort and assist vaginal preparation for delivery. In this embodiment, heating could be accomplished via a variety of mechanisms not limited to heated forced air and heating coils.

Additional Features:

In some embodiments, the shielding devices can be configured to facilitate patient heating and/or cooling. Accordingly, the shielding devices can include channels and/or outlets for temperature-controlled fluids such as air, water, and so on.

In some embodiments, the shielding devices can be configured to facilitate the display of images. For example, images can be projected onto the shielding devices or one or more image display screens can be integrated into the shielding devices. Such image display can provide images to assist the clinicians in the performance of the medical procedures.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A modular medical privacy garment, comprising:
a modular base garment with a waistband and two leg holes, the base garment defining an opening; and
a modular component that is attachable to the base garment to cover the opening, the modular component defining one or more openings.

2. The modular medical privacy garment of claim 1, further comprising:
an aseptic preparation kit that is integrated with the base garment; and
a cleanup kit that is integrated with the base garment.

3. The modular medical privacy garment of claim 1, wherein the modular component is shaped like a box.

4. The modular medical privacy garment of claim 3, wherein a peripheral wall of the box is pleated to allow extension and retraction of the box.

5. The modular medical privacy garment of claim 1, wherein the opening is located in an anterior region of the base garment.

6. The modular medical privacy garment of claim 1, wherein the opening is located in a posterior region of the base garment.

7. A shielding device adapted for use during a medical procedure, the shielding device comprising:
a shield body defining a proximal opening and a distal opening; and
an attachment area coupled to the shield body near the distal opening, the attachment area configured for releasably attaching the shielding device to a drape or gown.

8. The shielding device of claim 7, wherein the shield body is conical or frustoconical.

9. The shielding device of claim 7, wherein the shield body is cylindrical.

10. The shielding device of claim 7, further comprising a mechanism for selectively closing the proximal opening.

11. The shielding device of claim 7, wherein the attachment area comprises an adhesive.

12. A shielding device adapted for use during a medical procedure, the shielding device comprising:
a shield body defining a proximal opening and a distal opening;
an attachment mechanism disposed at the proximal opening, the attachment mechanism operable to releasably couple the shielding device to a shaft of a medical instrument; and
an attachment area coupled to the shield body near the distal opening.

13. The shielding device of claim 12, wherein the shield body is conical or frustoconical.

14. The shielding device of claim 12, wherein the shield body includes contoured lateral cut-outs.

15. The shielding device of claim 12, further comprising an umbrella mechanism attached to the shield body to make the shielding device radially expandable and retractable.

16. The shielding device of claim 12, wherein the shield body is circular.

17. The shielding device of claim 16, wherein the shield body comprises a plurality of generally triangular portions that slide with respect to each other like a fan.
